(19) United States
(12) Patent Application Publication
(10) Pub. No.: US 2025/0250627 A1
(43) Pub. Date: Aug. 7, 2025

(54) Method for Nucleic Acid Structure Analysis

(71) Applicant: SHIMADZU CORPORATION, Kyoto-shi (JP)

(72) Inventor: Yuko FUKUYAMA, Kyoto-shi (JP)

(21) Appl. No.: 18/870,542

(22) PCT Filed: May 31, 2023
(86) PCT No.: PCT/JP2023/020361

§ 371 Date: Nov. 29, 2024

(30) Foreign Application Priority Data
May 31, 2022 (JP) 2022-089236

(51) Int. Cl. C12Q 1/6872

(52) U.S. Cl. CPC C12Q 1/6872

(57) ABSTRACT

A method for nucleic acid structure analysis using an ion trap type mass spectrometer having an ion source performing MALDI includes: an ionization step of ionizing a nucleic acid contained in a sample by the ion source; an ion dissociation step of dissociating a protonated molecule or a deprotonated molecule of the nucleic acid generated in the ionization step by collision-induced dissociation inside an ion trap of the mass spectrometer to generate a plurality of fragment ions; a mass spectrometry step of performing mass spectrometry on the plurality of fragment ions generated in the ion dissociation step to acquire mass information of the plurality of fragment ions; and a structure determination step of determining at least a part of a structure of the nucleic acid based on the mass information of the plurality of fragment ions acquired in the mass spectrometry step.

### TECHNICAL FIELD

The present invention relates to a method for nucleic acid structure analysis using mass spectrometry.

### BACKGROUND ART

Nucleic acids are biopolymers in which nucleotides composed of a base, a sugar, and a phosphoric acid are linked by phosphodiester bonds, and are classified into deoxyribonucleic acid (DNA) and ribonucleic acid (RNA) depending on the difference in sugar. Among them, a nucleic acid in which several to about 20 nucleotides are polymerized, which may be called an oligonucleotide, can be chemically synthesized. Thus, research to apply an oligonucleotide as an oligonucleotide therapeutics has been actively conducted in recent years.

As a method for structural analysis of nucleic acids and oligonucleotides, a method using mass spectrometry is known. Here, the method for structural analysis of nucleic acids and oligonucleotides includes the method for identifying the binding order (base sequence) of nucleotides constituting DNA or RNA, identifying the type of chemical modification, or identifying the site subjected to the chemical modification. In this mass spectrometry method, a nucleic acid to be analyzed is intentionally fragmented, and various partial structures generated by the fragmentation are analyzed by mass spectrometry.

For example, Non Patent Literature 1 discloses a method of performing MS/MS analysis (also referred to as MS² analysis) using a tandem time-of-flight (TOF) mass spectrometer having an ion source performing a matrix-assisted laser desorption/ionization (MALDI) method (MALDI-TOF/TOF-MS). In the method, structural analysis of a nucleic acid having 4 nucleotides polymerized and a molecular weight of about 1200 is performed. Specifically, in a mass separation unit in a first stage, a protonated molecule ([M+H]⁺) of a nucleic acid to be analyzed is selected as a precursor ion, and the precursor ion is dissociated by collision-induced dissociation (CID) in a subsequent collision chamber to generate various fragment ions (also referred to as product ions). Then, the structural analysis is performed on the basis of mass information of fragment ions obtained by separating the various fragment ions in the mass separation unit in a further subsequent stage and detecting the fragment ions.

Since the MALDI method is generally a soft ionization method, ions hardly dissociate. However, it is known that dissociation of ions is promoted at the time of ionization by, for example, increasing the intensity of laser light to increase the ionization energy or using a special matrix. Such a method of dissociating ions simultaneously with, or immediately after, ionization is referred to as in-source decay (ISD). In the method described in Non Patent Literatures 2 to 4, various fragment ions of a nucleic acid generated by in-source decay are subjected to mass spectrometry using a time-of-flight mass spectrometer having an ion source performing the MALDI method (MALDI-TOF-MS), and the structural analysis of the nucleic acid is performed based on mass information of the fragment ions obtained by the mass spectrometry.

### CITATION LIST

### Non Patent Literature

Non Patent Literature 1: Thomas E. Andersen and two others, “RNA Fragmentation in MALDI Mass Spectrometry Studied by H/D-Exchange: Mechanisms of General Applicability to Nucleic Acids”, Journal of the American Society for Mass Spectrometry, (USA), 2006, 17, pp. 1353-1368
Non Patent Literature 2: Nathan A. Hagan and five others, “Enhanced In-Source Fragmentation in MALDI-TOF-MS of Oligonucleotides Using 1,5-Diaminonapthalene”, Journal of the American Society for Mass Spectrometry, (USA), 2012, 23, pp. 773-777
Non Patent Literature 3: Hisao Shimizu and six others, “Application of high-resolution ESI and MALDI mass spectrometry to metabolite profiling of small interfering RNA duplex”, Journal of Mass Spectrometry, (USA), 2012, 47, pp. 1015-1022
Non Patent Literature 4: Satoshi Kimura and one other, “Effect of oligonucleotide structural difference on matrix-assisted laser desorption/ionization in-source decay in comparison with collision-induced dissociation fragmentation”, (USA), Rapid Communications in Mass Spectrometry, 2020, 34, e8819
Non Patent Literature 5: Scott A. McLuckey and two others, “Tandem mass spectrometry of small, multiply-charged oligonucleotides”, Journal of the American Society for Mass Spectrometry, (USA), 1992, 3, pp. 60-70

### SUMMARY OF INVENTION

### Technical Problem

In order to perform structural analysis by MS/MS analysis using collision-induced dissociation as in Non Patent Literature 1, it is necessary to introduce a sufficient amount of protonated molecules ([M+H]⁺) or deprotonated molecules ([M−H]⁻) (M is a molecule, and H is a hydrogen atom) (hereafter, [M+H]⁺ and ([M−H]⁻) are called “molecular weight-related ions”) as precursor ions into a collision chamber in order to generate fragment ions. However, in mass spectrometry of a nucleic acid, the nucleic acid is easily decomposed at the time of ionization, and particularly, the larger the molecular weight of the nucleic acid is, the more easily the nucleic acid is decomposed, so that [M+H]⁺ or [M−H]⁻ is not easily generated. In addition, since an alkali metal ion adduct of a nucleic acid is easily generated besides [M+H]⁺ or [M−H]⁻, [M+H]⁺ or [M−H]⁻ tends to have low sensitivity. Therefore, it is difficult to introduce a sufficient amount of molecular weight-related ions into the collision chamber, and it may be difficult to perform structural analysis of the nucleic acid by MS/MS analysis. Further, in the structural analysis using the in-source decay as in Non Patent Literatures 2 to 4, there is a problem in that the resolution and sensitivity of some fragment ions to be detected tend to be low.

The present invention has been made in view of the above problem, and an object of the present invention is to provide a novel method for nucleic acid structure analysis capable of detecting fragment ions with high sensitivity.

### Solution to Problem

A method for nucleic acid structure analysis according to the present invention, which has been made to solve the above problems, is a method for nucleic acid structure analysis using an ion trap type mass spectrometer having an ion source performing a matrix-assisted laser desorption/ionization method, the method including:
an ionization step of ionizing a nucleic acid contained in a sample by the ion source;
an ion dissociation step of dissociating a protonated molecule or a deprotonated molecule of the nucleic acid generated in the ionization step by collision-induced dissociation inside an ion trap of the mass spectrometer to generate a plurality of fragment ions;
a mass spectrometry step of performing mass spectrometry on the plurality of fragment ions generated in the ion dissociation step to acquire mass information of the plurality of fragment ions; and
a structure determination step of determining at least a part of a structure of the nucleic acid based on the mass information of the plurality of fragment ions acquired in the mass spectrometry step.

### Advantageous Effects of Invention

As a result of intensive study, the inventor of the present application has obtained findings that when mass spectrometry of a nucleic acid is performed using an ion trap type mass spectrometer having an ion source performing the MALDI method, the influence of generation of an alkali metal ion adduct is suppressed, and as a result, [M+H]⁺ or [M−H]⁻ of the nucleic acid can be detected with high sensitivity. Based on this, MS² analysis of the nucleic acid by collision-induced dissociation was performed using the mass spectrometer, and it was found that fragment ions generated from [M+H]⁺ or [M−H]⁻ can also be detected with high sensitivity.

The collision-induced dissociation by the ion trap type mass spectrometer used in the present invention is called low-energy collision-induced dissociation (LE-CID) because kinetic energy at the time of collision between collision gas and target ions is relatively low at about 1000 eV (1 keV) or less (about several to several hundred eV). In the LE-CID, multiple collisions of low energy cause collision energy to accumulate as vibration energy in a sample molecule, and induces dissociation of ions. Since the accumulated energy is redistributed by reflecting the molecular structure, ions are dissociated from a position exceeding the limit point of the binding state. On the other hand, in high-energy collision-induced dissociation (HE-CID) in which collision energy is 1000 eV or more, most fragment ions are generated by one collision, and ions are dissociated mainly by simple cleavage occurring at a collision site. As described above, the LE-CID and the HE-CID have different ion dissociation mechanisms and different types of fragment ions to be obtained.

With the method for nucleic acid structure analysis according to the present invention, fragment ions can be detected with high sensitivity, and a novel method for nucleic acid structure analysis using an ion dissociation method different from a conventional method can be provided.

### BRIEF DESCRIPTION OF DRAWINGS

FIG. 1 A view showing a mass spectrum of a standard nucleic acid A obtained by MS analysis in a First Example.

FIG. 2 A view showing a product ion spectrum of a standard nucleic acid A obtained by MS² analysis in a First Example.

### DESCRIPTION OF EMBODIMENTS

Hereinafter, an embodiment of a method for nucleic acid structure analysis according to the present invention will be described.

### Nucleic Acid

For a nucleic acid to be analyzed according to the present embodiment, the number of nucleotides as a constitutional unit is not particularly limited, but an oligonucleotide in which several to several tens of nucleotides are linked is preferable. Among them, an oligonucleotide in which about 2 to 20 nucleotides are linked is preferable. In addition, the nucleic acid may be a natural product obtained from an organism or a processed product of the natural product, or may be a chemically synthesized artificial synthetic nucleic acid.

### Mass Spectrometer

In the present embodiment, an ion trap type mass spectrometer having an ion source performing the MALDI method is used. Specifically, a mass spectrometer including an ion source performing the MALDI method and an ion trap having a function of holding ions in the inside of the ion trap and separating ions according to their mass-to-charge ratios and a function of dissociating ions by collision-induced dissociation is used. In the mass spectrometer, not only analysis without ion dissociation (hereinafter, referred to as MS analysis) but also MSⁿ analysis in which selection and dissociation of ions are repeated one or more times (n is an integer of 2 or more) can be performed.

The ion trap type mass spectrometer herein has an ion trap for capturing ions generated by an ion source. Specifically, the ion trap type mass spectrometer includes a mass spectrometer configured to discharge ions captured in the ion trap in ascending order of mass-to-charge ratio (m/z) using a mass separation function of the ion trap itself, and detect the ions by a detector disposed outside the ion trap. In addition, the ion trap type mass spectrometer includes a mass spectrometer configured to separate ions simultaneously discharged from the ion trap in accordance with the mass-to-charge ratio by a mass separation unit disposed outside the ion trap, such as a time-of-flight mass analyzer for example, and detect the ions by a detector similarly disposed outside.

The type of the ion trap is not particularly limited. In the case of an RF trap that captures and discharges ions using a radio-frequency (Radio Frequency: RF) electric field, the RF trap may be an ion trap that captures ions using an electric field generated by applying a radio-frequency voltage of sinusoidal shape to a ring electrode, or may be a digital ion trap that captures ions using an electric field generated by applying a rectangular wave voltage generated by switching two different voltages at high speed to the ring electrode. In the digital ion trap, the range of m/z of ions that can be captured is controlled by changing the frequency while maintaining the amplitude (voltage value) of the rectangular wave voltage constant. A digital ion trap is preferably used as the ion trap.

A method for nucleic acid structure analysis according to the present embodiment includes: an ionization step of ionizing a nucleic acid contained in a sample with an ion source performing a MALDI method; an ion dissociation step of dissociating a protonated molecule or a deprotonated molecule of the nucleic acid generated in the ionization step inside an ion trap by collision-induced dissociation; a mass spectrometry step of performing mass spectrometry on a plurality of fragment ions derived from the nucleic acid generated in the ion dissociation step; and a structure determination step of determining a structure of the nucleic acid based on mass information of the plurality of fragment ions acquired in the mass spectrometry step.

### Ionization Step

In the ionization step, a sample for analysis containing a nucleic acid and a matrix substance is irradiated with laser light in an ion source performing the MALDI method to ionize the nucleic acid together with the matrix substance.

As the matrix substance, an appropriate substance can be selected according to the type of nucleic acid to be analyzed. Examples the matrix substance include 3-hydroxypicolinic acid (3-HPA), 2,4-dihydroxyacetophenone (2,4-DHAP), 2,5-dihydroxybenzoic acid (DHB), 2′,4′,6′-trihydroxyacetophenone monohydrate (THAP), 6-aza-2-thiothymine (ATT), 3-aminopyrazine-2-carboxylic acid (APCA), anthranilic acid (AA), and nicotinic acid (NA). Among them, preferably, 3-HPA, 2,4-DHAP, and THAP are used, more preferably, 3-HPA and 2,4-DHAP are used, and particularly preferably, 3-HPA is used. In addition, a mixed matrix in which two or more kinds of matrix substances are mixed may be used, and among them, preferably, a mixed matrix in which 3-HPA and 2,4-DHAP are mixed, a mixed matrix in which 3-HPA and THAP are mixed, and a mixed matrix in which 2,4-DHAP and THAP are mixed are used.

As a method for preparing a sample for analysis, a mixed solution in which a sample containing nucleic acid and a matrix substance are mixed is prepared, and the mixed solution is dried on a sample plate of a mass spectrometer. The mixed solution may be prepared in advance, and the mixed solution may be dripped onto a sample plate and dried, or the mixed solution may be prepared on a sample plate and dried as it is.

The sample for analysis may further contain a matrix additive. As the matrix additive, ammonium citrate dibasic (ACD) can be used. There are several types of ammonium salts of citric acid depending on the number of ammonium ions bonded to the citrate ion, and a salt in which two ammonium ions are bonded to one citrate ion is suitably used in the present embodiment.

When the sample for analysis contains the matrix additive, the order of mixing the sample containing the nucleic acid, the matrix substance, and the matrix additive is not particularly limited, but it is preferable that a matrix-additive mixed solution containing the matrix substance and the matrix additive is prepared in advance, and the sample solution containing the nucleic acid and the matrix-additive mixed solution are mixed to prepare the sample for analysis. In this case, a mixed solution obtained by mixing the sample solution and the matrix-additive mixed solution in advance may be dripped onto a sample plate and dried to prepare a sample for analysis, or the sample solution and the matrix-additive mixed solution may be dripped onto a sample plate and mixed on the sample plate and dried. The ratio of mixing the sample solution and the matrix-additive mixed solution is not particularly limited. By preparing the matrix-additive mixed solution in advance, it is easy to prepare a sample for analysis. Preferably, the concentration of the matrix additive in the matrix-additive mixed solution is 10 to 100 mM, and more preferably 30 to 70 mM, from the viewpoint of generating a sufficient amount of molecular weight-related ions of the nucleic acid. In the present specification, a numerical range from a lower limit value to an upper limit value is indicated as “(lower limit value) to (upper limit value)” using the word “to”, and the numerical range indicated in this manner includes the lower limit value itself and the upper limit value itself.

### Ion Dissociation Step

In the ion dissociation step, first, all ions generated by the ionization step are temporarily captured in an ion trap, ions other than a protonated molecule ([M+H]⁺) or a deprotonated molecule ([M−H]⁻) of the nucleic acid to be analyzed are discharged from the ion trap, and the protonated molecule or the deprotonated molecule is selected as a precursor ion. Next, for example, an inert gas such as argon is introduced into the ion trap, and protonated molecules or deprotonated molecules are dissociated by collision-induced dissociation. Thus, various fragment ions (product ions) derived from nucleic acid are generated.

The collision-induced dissociation in the ion trap type mass spectrometer used in the present embodiment corresponds to low-energy collision-induced dissociation (LE-CID). In the LE-CID, collision energy is accumulated as vibration energy in a sample molecule by multiple collision to induce dissociation of ions, and the accumulated energy is redistributed reflecting a molecular structure, so that ions are dissociated from a position exceeding a limit point of a binding state of the molecule. On the other hand, collision-induced dissociation in a time-of-flight mass spectrometer corresponds to high-energy collision-induced dissociation (HE-CID), and ions are simply dissociated at the collision point. Thus, the type of CID (LE-CID or HE-CID) differs depending on the mass spectrometer used, and the LE-CID differs from the HE-CID in the manner of ion dissociation.

### Mass Spectrometry Step

In the mass spectrometry step, various fragment ions derived from the nucleic acid generated in the ion dissociation step are subjected to mass spectrometry (MS² analysis). When the mass spectrometer used in the present embodiment utilizes the mass separation function of the ion trap itself, fragment ions are discharged from the ion trap in ascending order of mass-to-charge ratio, and the discharged ions are detected by a detector disposed outside the ion trap to perform mass spectrometry. When the mass spectrometer utilizes a mass separation function of a mass separation unit that is not an ion trap, various fragment ions simultaneously discharged from the ion trap are introduced into a mass separator disposed outside the ion trap to separate the ions according to the mass-to-charge ratio, and the separated fragment ions are detected by a detector similarly disposed outside the ion trap to perform mass spectrometry. By performing MS² analysis, the mass spectrum (product ion spectrum) of various fragment ions can be obtained. As described above, since the LE-CID and the HE-CID have different ion dissociation manners, the MS² analysis using the LE-CID by the ion trap type mass spectrometer can obtain a product ion spectrum different from that of the MS² analysis using the HE-CID.

### Structure Determination Step

In the structure determination step, peaks corresponding to various fragment ions are extracted from the mass spectrum obtained in the mass spectrometry step, various fragment ions are assigned based on the mass-to-charge ratio (mass information) indicated by the peaks, and the results are combined to determine at least a part of the structure of the original nucleic acid. The determination of the structure includes sequence analysis and specifying the type of chemical modification or the site subjected to the chemical modification by the sequence analysis. Database search or De Novo Sequencing may be used to determine the structure.

Hereinafter, a method for nucleic acid structure analysis according to the present invention will be described by way of example, but this is merely an example, and the present invention is not limited to this.

### EXAMPLES

### 1. Preparation of Sample Solution

As a sample solution, a 100-pmol/μL aqueous solution of standard nucleic acid A (5′-CAATGTGC-3′: MW 2409.6,) was prepared.

### 2. Preparation of Matrix-Additive Mixed Solution

As a matrix-additive mixed solution, a 40-mg/mL 50% acetonitrile (ACN) aqueous solution of 3-hydroxypicolinic acid (3-HPA) containing ammonium citrate dibasic (ACD) at a concentration of 40 mM as a matrix additive was prepared.

### 3. Preparation of Sample for Analysis

The sample solution prepared in 1. and the matrix-additive mixed solution prepared in 2. were mixed at a ratio of 1:1 (v/v), and 1 μL of the obtained mixed solution was dripped onto a sample plate (SUS plate) and dried.

### 4. Mass Spectrometry

For the mass spectrometry, a MALDI digital ion trap type mass spectrometer (MALDI-DITMS, manufactured by Shimadzu Corporation, trade name: MALDImini-1) was used. The sample for analysis prepared in 3. was introduced into MALDI-DITMS, and MS analysis and MS² analysis were performed in positive mode.

### 5. Results

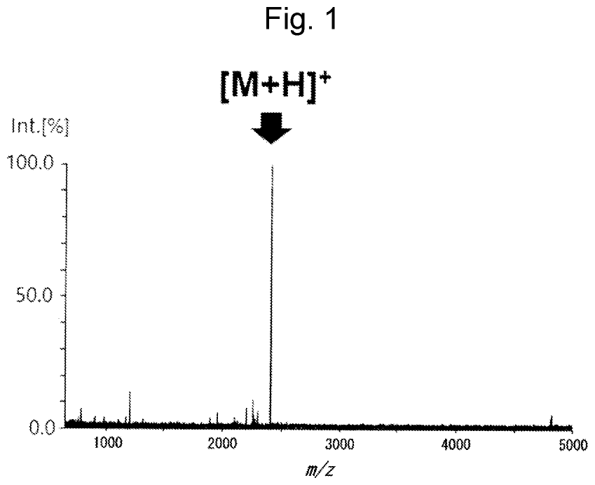

FIG. 1 shows a mass spectrum obtained by the MS analysis. The arrow in the figure indicates the detection status of a protonated molecule ([M+H]⁺). From FIG. 1, it has been confirmed that [M+H]⁺ is detected with high sensitivity.

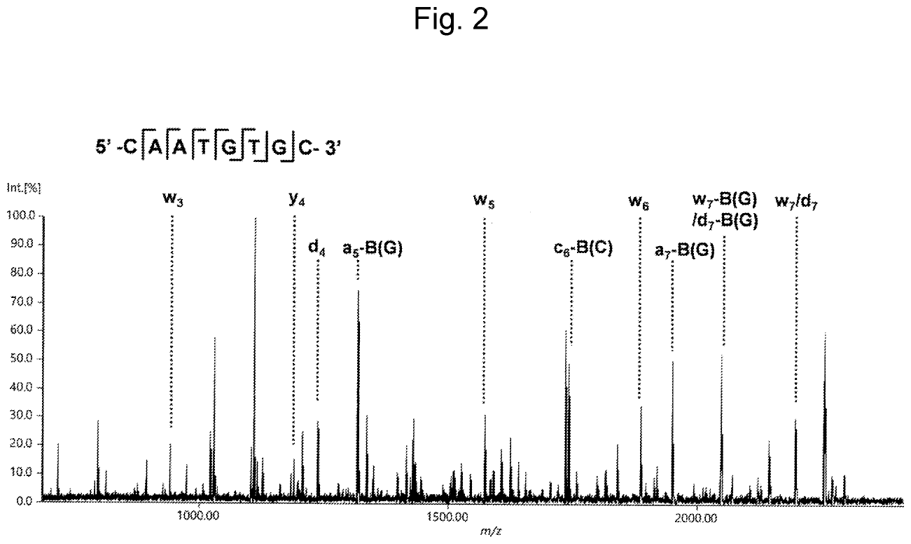

FIG. 2 shows a product ion spectrum obtained by the MS² analysis with respect to [M+H]⁺. In FIG. 2, each peak in the mass spectrum is given the name of the fragment ion species of the corresponding oligonucleotide (general name proposed in Non Patent Literature 5). The name is a name representing respective ion species in a fragment ion sequence according to a nucleic acid dissociation pattern naming rule. In this naming rule, fragment ions including the 5′-end are denoted as aₙ, bₙ, cₙ, and dₙ, and fragment ions at the 3′-end in the opposite direction are denoted as xₘ, yₘ, zₘ, and wₘ. Note that the suffixes n and m indicate the number of constitutional units (the number of bases by definition) from the corresponding end to the dissociation site. From FIG. 2, peaks of various fragment ions were detected with high sensitivity, and on the basis of the w-series ions, fragment ions of sufficient types to perform structural analysis were able to be assigned, and the entire base sequence of the standard nucleic acid A was able to be analyzed.

### Modes

It will be understood by those skilled in the art that the exemplary embodiments described above are specific examples of the following modes.

### (Clause 1)

A method for nucleic acid structure analysis according to a mode of the present invention is a method for nucleic acid structure analysis using an ion trap type mass spectrometer having an ion source performing a matrix-assisted laser desorption/ionization method, the method including:
an ionization step of ionizing a nucleic acid contained in a sample by the ion source;
an ion dissociation step of dissociating a protonated molecule or a deprotonated molecule of the nucleic acid generated in the ionization step by collision-induced dissociation inside an ion trap of the mass spectrometer to generate a plurality of fragment ions;
a mass spectrometry step of performing mass spectrometry on the plurality of fragment ions generated in the ion dissociation step to acquire mass information of the plurality of fragment ions; and
a structure determination step of determining at least a part of a structure of the nucleic acid based on the mass information of the plurality of fragment ions acquired in the mass spectrometry step.

Thus, a novel method for nucleic acid structure analysis capable of detecting fragment ions with high sensitivity can be provided.

### (Clause 2)

In the method for nucleic acid structure analysis according to clause 1,
the ionization step may be performed by irradiating a sample for analysis containing a sample containing the nucleic acid, a matrix substance, and a matrix additive that is ammonium citrate dibasic with laser light.

Thus, more protonated molecules or deprotonated molecules of nucleic acid are generated, and fragment ions can be detected with higher sensitivity.

### (Clause 3)

In the method for nucleic acid structure analysis according to clause 2,
the matrix substance may be 3-hydroxypicolinic acid.

Thus, more protonated molecules or deprotonated molecules of nucleic acid are generated, and fragment ions can be detected with higher sensitivity.

### (Clause 4)

In the method for nucleic acid structure analysis according to clause 2 or 3,
when a matrix-additive mixed solution containing the matrix substance and the matrix additive is prepared, concentration of the matrix additive in the matrix-additive mixed solution may be 10 to 100 mM.

Thus, more protonated molecules or deprotonated molecules of nucleic acid are generated, and fragment ions can be detected with higher sensitivity.

### (Clause 5)

In the method for nucleic acid structure analysis according to clauses 2 to 4,
a matrix-additive mixed solution containing the matrix substance and the matrix additive may be prepared in advance, and
the sample for analysis may be prepared by mixing the sample and the matrix-additive mixed solution.

Thus, a sample for analysis can be easily prepared.

### (Clause 6)

In the method for nucleic acid structure analysis according to any one of clauses 1 to 5,
the mass spectrometer may be a digital ion trap type mass spectrometer.

Thus, more protonated molecules or deprotonated molecules of nucleic acid are generated, and fragment ions can be detected with higher sensitivity.

1. A method for nucleic acid structure analysis using an ion trap type mass spectrometer having an ion source performing a matrix-assisted laser desorption/ionization method, the method comprising:
an ionization step of ionizing a nucleic acid contained in a sample by the ion source;
an ion dissociation step of dissociating a protonated molecule or a deprotonated molecule of the nucleic acid generated in the ionization step by collision-induced dissociation inside an ion trap of the mass spectrometer to generate a plurality of fragment ions;
a mass spectrometry step of performing mass spectrometry on the plurality of fragment ions generated in the ion dissociation step to acquire mass information of the plurality of fragment ions; and
a structure determination step of determining at least a part of a structure of the nucleic acid based on the mass information of the plurality of fragment ions acquired in the mass spectrometry step.

2. The method for nucleic acid structure analysis according to claim 1, wherein the ionization step is performed by irradiating a sample for analysis containing a sample containing the nucleic acid, a matrix substance, and a matrix additive that is ammonium citrate dibasic with laser light.

3. The method for nucleic acid structure analysis according to claim 2, wherein the matrix substance is 3-hydroxypicolinic acid.

4. The method for nucleic acid structure analysis according to claim 2, wherein when a matrix-additive mixed solution containing the matrix substance and the matrix additive is prepared, concentration of the matrix additive in the matrix-additive mixed solution is 10 to 100 mM.

5. The method for nucleic acid structure analysis according to claim 2,
wherein a matrix-additive mixed solution containing the matrix substance and the matrix additive is prepared in advance, and
the sample for analysis is prepared by mixing the sample and the matrix-additive mixed solution.

6. The method for nucleic acid structure analysis according to claim 1, wherein the mass spectrometer is a digital ion trap type mass spectrometer.
